(19) United States
(12) Patent Application Publication
(10) Pub. No.: US 2023/0012295 A1
(43) Pub. Date: Jan. 12, 2023

(54) METHOD OF GENERATING CREDIBLE SOLUTIONS FROM NON-VALIDATED DATASETS

(71) Applicant: MEDXTRACTOR CORP., Calgary (CA)

(72) Inventor: James Morris DURWARD, Calgary (CA)

(21) Appl. No.: 17/848,962

(22) Filed: Jun. 24, 2022

Related U.S. Application Data
(60) Provisional application No. 63/220,004, filed on Jul. 9, 2021.

(51) Int. Cl. G16H 50/30; G16H 10/20; G16H 10/60; A61B 5/00; A61B 5/372

(52) U.S. Cl. CPC G16H 50/30; G16H 10/20; G16H 10/60; A61B 5/7267; A61B 5/372

(57) ABSTRACT

A method for calculating a risk of developing a medical condition includes receiving results of a biometric questionnaire of a patient, scoring said results and providing the scored results to a machine learning (ML) engine. The ML engine correlates the scored results with new non-scored results to calculate a probability of the patient developing a medical condition. Correlations are provided for various age and sex groups in order to predict longitudinal illness development.

### CROSS REFERENCE TO RELATED APPLICATIONS

This application claims the benefit of priority to U.S. Provisional Application Ser. No. 63/220,004 filed Jul. 9, 2021, the contents of which are hereby incorporated by reference.

### FIELD OF THE INVENTION

The present invention relates to a process of problem solving and the limitations of problem solving based on personal knowledge and the correlation of multiple, non-validated datasets. In particular, the present invention pertains to a method to effectively expand both a person's knowledge and correlation ability in order to increase the probability of determining an optimum solution to a problem.

### BACKGROUND

All people have a “scope” that may be viewed as a combination of their personal experiences, learned bias and interpretation ability. They rely on their scope when forming opinions and solutions to problems. While some people, often referred to as “experts”, have more comprehensive scopes than regular people, even experts are limited in scope when there are increasing demands on their time and new advances occurring daily. Even the highest performance people have limits.

While there are numerous diverse databases containing a multitude of data records, the formats and records are most often, respectively, incompatible, or incomplete. To exacerbate the problem, the data records often include metadata, description data that describes the data records, which may be irrelevant for some applications. Trying to combine databases often leads to a highly fractured situation that has evolved primarily due to the nature of basic research, where different databases have been created for different purposes. There have been, and continues to be, attempts to standardize data formats and metadata fields but these have been and are being stymied due to a variety of factors including, but not limited to intellectual property issues, privacy concerns, political issues, incompatible agendas, and a narrow or divergent focus.

Electroencephalogram (EEG) is a medical analysis technique where electrodes are attached to a patient's scalp and used to detect electrical activity in your brain. EEGs may be used to diagnose medical disorders such as brain tumors, head injuries, encephalopathy, encephalitis, stroke, sleep disorders and other conditions. EEG results may also be used to predict patient susceptibility to some mental conditions over their lifetime.

The process for gathering EEG data and combining the measured results with various disparate databases is time consuming and prone to error as the process relies on the scope of experts in order to obtain useful results. Therefore, there exists a need for a process to gather patient data and to generate a high-probability predictions to any number of potential medical conditions that obviates or mitigates one or more limitations of the prior art.

This background information is provided to reveal information believed by the applicant to be of possible relevance to the present invention. No admission is necessarily intended, nor should be construed, that any of the preceding information constitutes prior art against the present invention.

### SUMMARY

An object of embodiments of the present invention is to provide a system and method that provides for the collection of biometric patient data, which data includes EEG data, into a database to train an expert system that predicts the probability of the existence of current mental or physical illnesses or conditions as well as the patient's probability of developing particular mental and physical illnesses at a later date.

In accordance with embodiments of the present invention, there is provided a method for calculating a probability of developing a medical condition. The method includes receiving results of a biometric questionnaire of a patient, scoring the biometric data using digitized opinion algorithms based on the opinions of specialist experts, providing the results to a machine learning (ML) engine, creating a correlated training set from the (ML) engine, having the (ML) engine correlate a patient's biometric data that does not include EEG data against the training set, receiving predicted probabilities of developing the medical condition from the (ML) engine, and reporting to the patient for display in a variety of reports the probability of an existing condition and the probability of developing a condition at a future time.

In further embodiments, the probability is calculated by comparing the EEG estimates to baseline values.

In further embodiments, the baseline values are selected based on the patient's age, sex, or other biometric qualifiers.

Embodiments further include calculating a second probability of the patient developing the medical condition where the second probability is based on comparing baseline values corresponding to a future age of the patient. The second probability predicts a risk of the patient developing the medical condition at a future time.

Embodiments further include using the probability and the second probability to produce a timeline of a risk of the patient developing the medical condition over a time span.

Embodiments further include generating a report indicating the patient's risk of developing the medical condition at a future date.

In further embodiments, the report may include suggestions on a biometric change the patient can make to reduce the patient's risk of developing the medical condition at a future date.

In accordance with embodiments of the present invention, there is provided a method of training a machine learning (ML) engine. The method includes receiving biometric results of a biometric questionnaire of a patient, receiving electroencephalogram (EEG) results of the patient, trimming the EEG results to obtain stable EEG results, converting the EEG results into a plurality of summary values, combining the plurality of summary values with the biometric results to produce a record, scoring the record based on expert opinions, providing the scored record to a machine learning (ML) engine, receiving predictions from the ML engine, comparing the predictions to the expected predictions to generate an error, and utilizing the error to adjust coefficients of the ML engine.

Further embodiments include repeating the method until the error is below a threshold.

In further embodiments, the summary values are used to produce baseline values when the ML engine is used in a prediction mode.

In further embodiments, the baseline values are associated with an age, sex, and possibly other biometric variables of the patient.

In further embodiments, the trimming of the EEG results comprises discarding a last portion of the EEG results and discarding a first portion of the remaining EEG results.

Embodiments have been described above in conjunctions with aspects of the present invention upon which they can be implemented. Those skilled in the art will appreciate that embodiments may be implemented in conjunction with the aspect with which they are described but may also be implemented with other embodiments of that aspect. When embodiments are mutually exclusive, or are otherwise incompatible with each other, it will be apparent to those skilled in the art. Some embodiments may be described in relation to one aspect, but may also be applicable to other aspects, as will be apparent to those of skill in the art.

### BRIEF DESCRIPTION OF THE FIGURES

Further features and advantages of the present invention will become apparent from the following detailed description, taken in combination with the appended drawings, in which:

FIG. 1 illustrates a general method for training an expert system, according to an embodiment.

FIG. 2 illustrates a general method for using a trained expert system to predict the risk of a patient developing a medical condition, according to an embodiment.

FIG. 3 illustrates a method to collect EEG data from a group of baseline patients of a particular age group and sex, according to an embodiment.

FIG. 4 illustrates a detailed method to create and update a training set, according to an embodiment.

FIG. 5 illustrates a method of incorporating EEG data into a training set, according to an embodiment.

FIG. 6 illustrates a set of average EEG values used by embodiments.

FIG. 7 illustrates how subsequent average values may be derived from EEG measurements, according to an embodiment.

FIG. 8 illustrates how EEG derived values may be correlated with the medical conditions of interest, according to an embodiment.

FIG. 9 illustrates an example of a patient report as generated by embodiments.

FIG. 10 illustrates an example of a patient report as generated by embodiments for the patient of FIG. 9, according to an embodiment.

FIG. 11 depicts the components of the validated information collection, according to an embodiment.

FIG. 12 depicts and embodiment illustrating how scored records including both EEG, predicted or real, and biometric data may be processed, according to an embodiment.

FIG. 13 depicts an embodiment where a number of incoming records are input to an artificial intelligence/machine engine for re-analysis and re-scoring, according to an embodiment.

FIG. 14 depicts an embodiment where an individual record being resubmitted to a conditioning and addition algorithm.

It will be noted that throughout the appended drawings, like features are identified by like reference numerals.

### DETAILED DESCRIPTION

The terminology used herein is for the purpose of describing particular embodiments only and is not intended to be limiting of the invention. As used herein, the singular forms “a”, “an”, and “the” are intended to include the plural forms as well as the singular forms, unless the context clearly indicates otherwise. It will be further understood that the terms “comprises” or “comprising” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly-used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, and for the sake of clarity, this description shall refrain from repeating every possible combination of the individual steps in an unnecessary fashion. Nevertheless, the specification and claims should be read with the understanding that such combinations are entirely within the scope of the invention and the claims.

New approaches to the problem are discussed herein. In the following description, for purposes of explanation, numerous specific details are set forth in order to provide a thorough understanding of the present invention. The present invention will now be described by referencing the appended figures representing preferred embodiments.

Embodiments include methods that use biometric data to predict how a patient's health may develop over time based on genetic expressions, how they choose to live their life, their lifestyle, and their life experiences. The methods correlate patient lifestyle changes over time and place a patient's current situation in a timeline. The patient can change lifestyle variables and see how the predictions change.

Embodiments include a trained artificial intelligence/machine learning (AI/ML) system where a patient inputs biometric data to an online questionnaire, an ML machine correlates the questionnaire results with a training set of baseline data, to predict probabilities of the patient having or having a probability of developing any number of medical conditions. Based on the predictions, a graphical report may be generated for the patient. The graphical report can show a predicted timeline of the onset of medical conditions, and various changes that the patient may make to move the probabilities toward baseline “normal”, thereby lessening the chance of developing the medical conditions.

Embodiments of the present invention provide methods and systems to predict a probability of a patient developing a medical condition based on responses to a biometric questionnaire completed by the patient. As used herein, “medical condition” or “condition” refers to any physical or mental illness, condition, or affliction where the probability of developing that condition is correlated with EEG results. In embodiments, medical condition includes depressive disorders, anxiety disorders, trauma-related disorders, manic disorders, possible ADHD, possible OCD, Parkinson's disease, learning disabilities and bi-polar disorder. Though these are all mental conditions, embodiments may also apply to physical illnesses. Medical conditions that are later found to be correlated with EEG results may also be predicted using embodiments.

The system utilizes a trained expert system that is based on a training set that is generated by correlating baseline scores with collected patient biometric data. In use, a patient completes a questionnaire, a computer system machine correlates the questionnaire responses with the baseline training set, and may generate a graphic report for the patient, their care giver, or other party. The report may include a predicted timeline of illness or condition onset and various suggestions for the patient to adjust their probability score towards a “normal” baseline, thereby reducing the chance that the patient develops one or more medical conditions at a later date.

Embodiments comprise computer instructions and software stored on a non-transitory computer readable medium or memory that when executed by computer hardware, performs the methods described herein, as is known in the art. The computer software may be organized in any number of files, modules, functions, etc. The computer hardware may be one or more physical of virtual machines and may be located at a single location, a server location, on cloud computing infrastructures, etc. Each computer hardware device includes one or more processors, non-transitory computer memory, and wired or wireless network connections as required to communicate between computer nodes or locations.

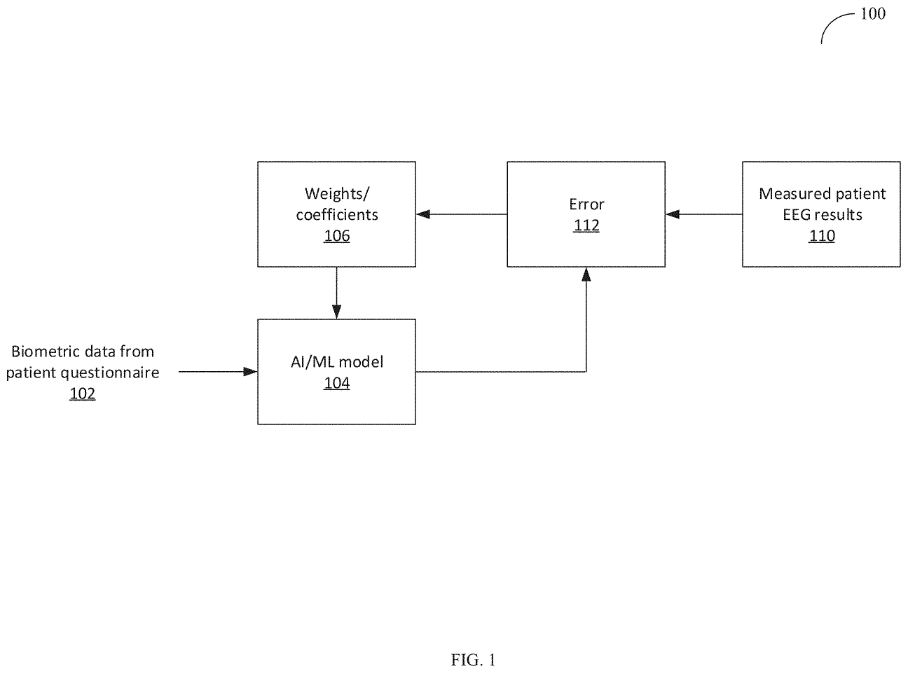

FIG. 1 illustrates a general method 100 for training an expert system including an artificial intelligent/machine (AI/ML) learning model according to an embodiment. A number of baseline patients are selected or recruited and divided into groups based on age and sex but may also include other variables. The number of baseline patients in each baseline group is sufficiently large to provide statistically significant results. In embodiments, there may be at least 20 patients in each age/sex group.

Biometric data 102 is obtained from each patient through the use of a questionnaire which provides inputs to the AI/ML model 104. The questionnaire includes questions based on the patient's genetic expressions, historical situation, recent situation and the present situation. The patient's historical situation is based on formative events generally between the ages of 2 and 20 years of age that are not expected to change should a patient respond to the questionnaire multiple times. An example of a historical situation would be a traumatic event that may have happened when the patient was a child, or their academic history. The patient's recent situation may include occurrences within approximately the past 5 years such as smoking and/or cessation, and present situation may be based on events happening at present or within approximately the past year. An example of a present situation would be an amount of alcohol consumed or whether they have full time employment. In embodiments, a patient may complete the biometric questionnaire multiple times while only completing the present situation questions on the second and subsequent times.

The AI/ML model 104 also receives weights or coefficients to configure the AI/ML model 104. The AU/ML model 104 may output a set of predicted EEG results for each baseline patient. During training, each baseline patient also has an EEG scan performed that generated measured EEG results 110. Comparing the predicted EEG results with the patient's measured EEG results 110 generates an error which is fed back to determine updated weights 106. The process is repeated until there is convergence on the AI/ML model 104 and the error is sufficiently small. The training process allows for the AI/ML model 104 to determine correlations between the questionnaire data and EEG results in order to produce predicted illnesses 202

In embodiments, probabilities of a patient developing a medical condition are determined. Furthermore, correlations between sets of questionnaire questions are also determined. This allows predictions to be made between questionnaire questions and probabilities of a patient developing a medical condition and the identification of which questions most influence the probabilities of developing a particular medical condition.

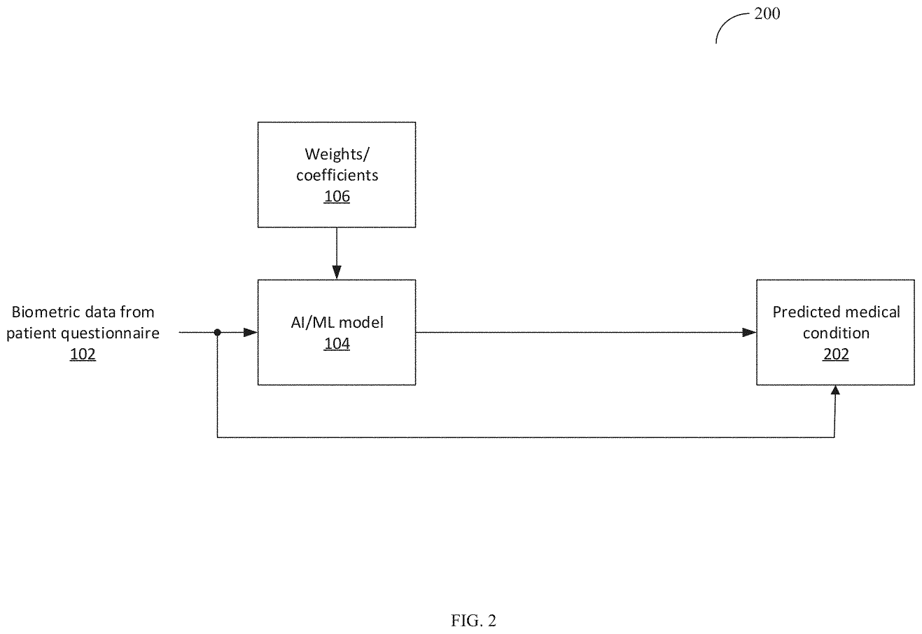

FIG. 2 illustrates a general method 200 for using a trained expert system to predict the risk of a patient developing medical conditions at a later date. Biometric data 102 is obtained from patient questionnaires which provide inputs to the AI/ML model 104. The AI/ML model 104 also receives weights or coefficients to configure the AU/ML model 104. The AI/ML model 104 outputs a set of predicted results 202 for the patient. The predicted results are used to predict medical conditions 202, which may be displayed with a variety of common data visualization methods. With a trained AI/ML system, the predicted medical conditions 202 are obtained without requiring EEG measurements from the patient.

Medical condition EEG signatures are known in the art and there are numerous studies that indicate correlations between EEG signatures and medical conditions with common conclusions in this regard. In embodiments, these EEG signatures are used in a scoring algorithm for each condition from which predictions may be made. For example, when the differential from EEG baseline concurrently shows elevated delta measurements, high frontal alpha measurements, high right beta measurements, and elevated right theta measurements, obsessive-compulsive disorder (OCD) may be indicated. When baseline differentials include theta measurements above average, a measurements low beta/theta differential, and beta measurements below average, Anxiety Depression may be indicated. The greater the overall differential between measured or predicted EEG results and baseline, normal results, the more probable that the patient may suffer from the medical condition.

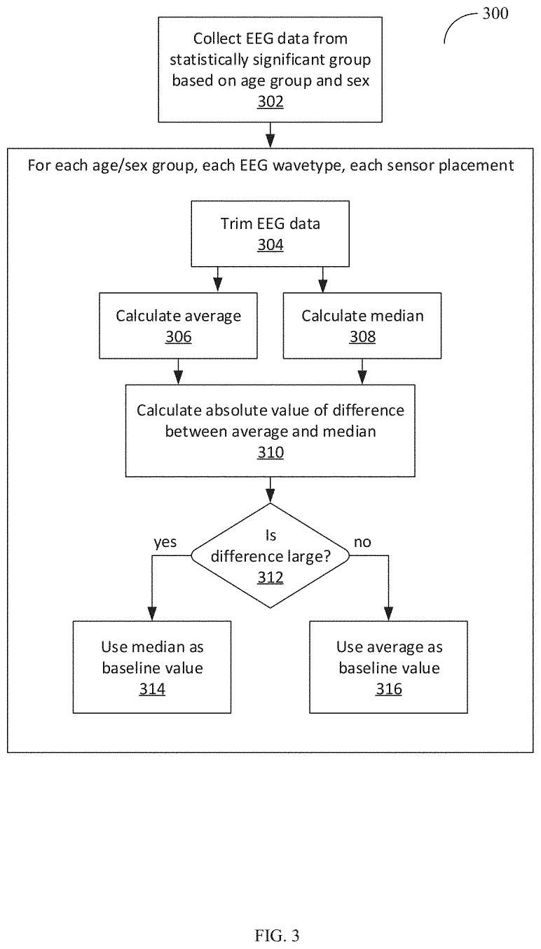

FIG. 3. Illustrates a method to collect EEG data from a group of baseline patients. Baseline patients are divided into groups based on age, sex, and possibly other variables. For example, baseline patients may be divided into four age groups as well as male and female, for a total of 8 different groups. More or less age groups may also be used. Furthermore, groups may be formed using other socioeconomic factors. Each group will be used to form its own reference baselines (RB) as required. Each of the age and sex groups (8 different baselines in this example) will have their own RBs which are meant to represent a “normal” median value for that group. When generating the training set, baseline participation should be limited to patients with no known mental or physical conditions that may influence the results. In embodiments, the baseline participants should have little or no occurrence of the medical conditions being predicted. Additionally, patients should ideally only drink occasionally or not at all, and shouldn't consume recreational drugs, should not be obese and should have no physical, mental or sexual illnesses or traumas. In order generate statistically significant results, the RBs for each group should include a minimum of 20 participants. As more participants are added over time, the RBs may be re-calculated to increase the resolution and provide for changing baselines going forward.

In embodiments, age groups are established based on age ranges such as 18-29 years old, 30-44 years old, 45-55 years old, and 56-80 years old, though other ranges, or a greater or lesser number of ranges are possible. Baseline patient groups are also divided by sex; male or female. Embodiments may use other groupings as well where correlations between EEG results and medical conditions are related to these other groupings.

In step 302 EEG data is collected from each patient in an age/sex group. EEG readings involve the placement of electrodes on the head of the patient and electrical waveforms of one or more wave types recorded. In embodiments, four electrodes are placed on the patient; right frontal, left frontal, right temporal, and left temporal. Five wave types are recorded; delta, theta, alpha, beta, and gamma. Recorded readings are digitized over the time of the recording and may be saved as x-y coordinates where x is time and y is a negative or positive value of the recorded brain wave. Recording sessions take place over a period of time, such as 15 minutes. In order to obtain stable readings, some readings are trimmed and discarded in step 304. In embodiments, a last portion of the recording is discarded and a first portion of the recording minus the last minute is also discarded. As examples, for a 15 minute recording session, the last minute and the first seven minutes are discarded. For a 25 minute recording session, the last minute and the first 12 minutes are discarded. This results in a trimmed set of x-y values for each wave type of each patient in the age/sex group. In step 306, an average value (y value) for each wave type is calculated. In step 308, a median of the y values for each wave type is calculated. In order to calculate the most useful of the average or median, in step 310, the absolute value of the difference between the average value of step 306 and the median value of step 308 is calculated. If the difference is large (step 312), the median will be used as a baseline value (step 314). In embodiments, a difference greater than 0.03 is a large difference, and the average value will be used. The method of FIG. 3 results in a baseline value for each age/sex group for each EEG wave type for each electrode reading.

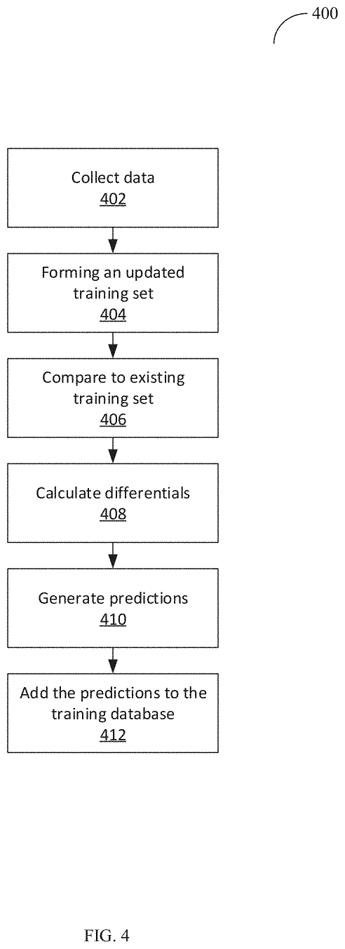

With reference to FIG. 4 which illustrates a detailed method 400 to create and update a training set, according to an embodiment. Method 400 includes the steps of gathering data 402, forming an updated training set 404, comparing the newly gathered data to the existing training set 406, calculating derived values for all data elements against all other data elements 408, generating algorithmically-scored predictions 410, and adding the scored predictions to the training database to increase its size and accuracy 412.

The training set includes a Reference Database (RF) and is formed from Reference Records (RRs) consisting of biometric data and electroencephalogram (EEG) data. Biometric data is collected with a questionnaire while EEG data is collected using the method 300 of FIG. 3. In step 402, biometric data is gathered using a questionnaire, which in embodiments may be an online or computer implemented questionnaire. A patient, reference candidate, or any other person for whom results are desired, provides biometric data to complete the questionnaire. In an embodiment there may be between 175 and 250 questions to collect biometric data covering a variety of life development events. The biometric data may include:
Basic data such as age, sex, or gender.
Inherited features that are DNA “expressions” such as hair colour, blood type, aptitudes, etc.
Information from the patient's childhood and formative years between the approximate ages of 2 and 20. This information includes events such as traumas, phobias, and allergies.
Information related to the patient's present situation such as significant events that have occurred during the past year.
Diagnosed mental and physical illnesses (mental and physical) in the past or within the past year.
Prescribed drugs within the past year.
Family medical history such as illnesses.
General background in the past as well as the past year.

Questionnaire answers are recorded as numerical values and later combined with EEG data to form reference records (“RR”s). If included, contact information may be stripped to preserve anonymity and the final RR may be represented as a single row record, which may be up to 2000 columns wide. RRs are saved to a database (such as an SQL database within Microsoft's “AZURE” cloud platform, or other platform) to create a reference database used for training the AI/ML engine 104.

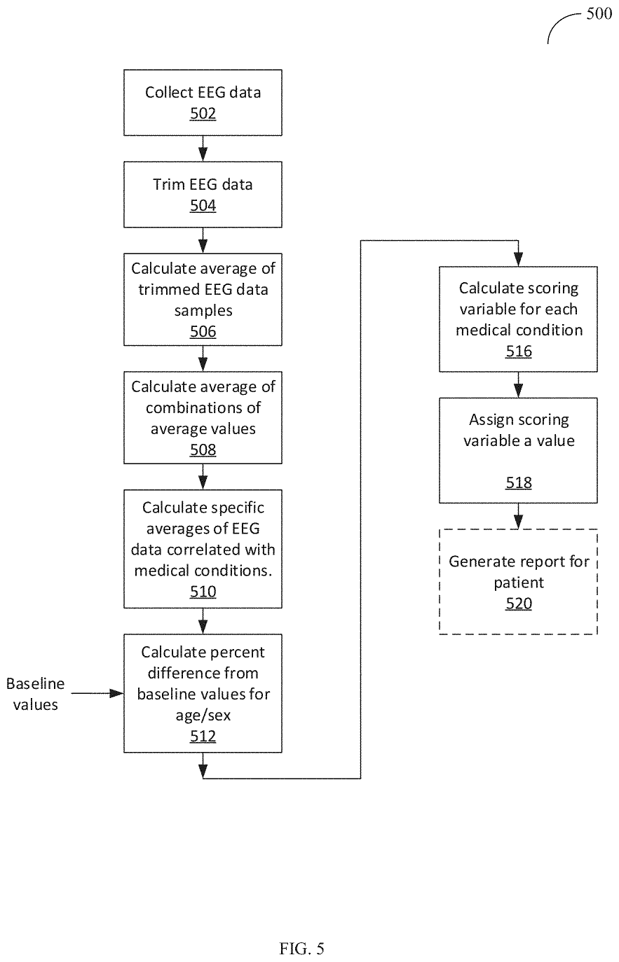

FIG. 5 illustrates a method 500 of incorporating EEG data into a training set, according to an embodiment. In step 502 EEG data may be collected in a session conducted in a calm, quiet, or silent location, with the patient's eyes closed, over an approximately 15 minute period in order to capture the patient's brain waves without audio-visual distraction. In embodiments, EEG sessions are conducted at approximately the same time of day, for example between 9:00 am and 11:30 am, to help obtain consistent readings across EEG readings from different patients. An EEG technician administering the EEG test may visually inspects all EEG files before uploading the results to a collection, such as an email box, folder or other storage space. In embodiments, it may be sufficient to record only the right, and left frontal and right and left temporal lobe readings when collecting data for the training set as these are the primary lobes known in the literature to be involved in medical conditions, such as some mental illnesses. In other cases, a full set of EEG readings should be collected as is known in the art. EEG data is collected for wave types of interest which in embodiments includes five wave types; delta, theta, alpha, beta, and gamma.

EEG data is stored as a series of x-y coordinates where x is time and y is a negative or positive value of the recorded brain wave. In step 504 the digitized EEG data is trimmed to discard the last minute of the recording, and the first half of the recording minus the last minute. For example, for a 15 minute recording session, the last minute and the first seven minutes are discarded. For a 25 minute recording session, the last minute and the first 12 minutes are discarded.

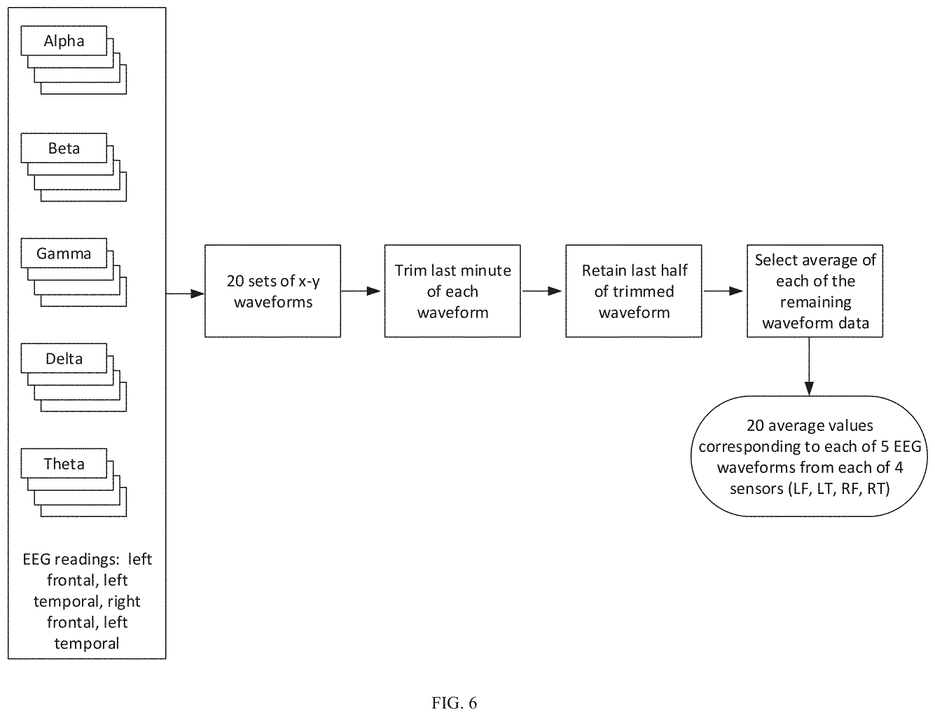

In step 506, the average of the trimmed EEG y values is calculated for each electrode of each wave types being used. In embodiment, four sensors and five wave types will result in 20 separate average EEG values. Alternatively, the median of the EEG values may be use. The process of trimming EEG values and obtaining a set of average values is illustrated in FIG. 6.

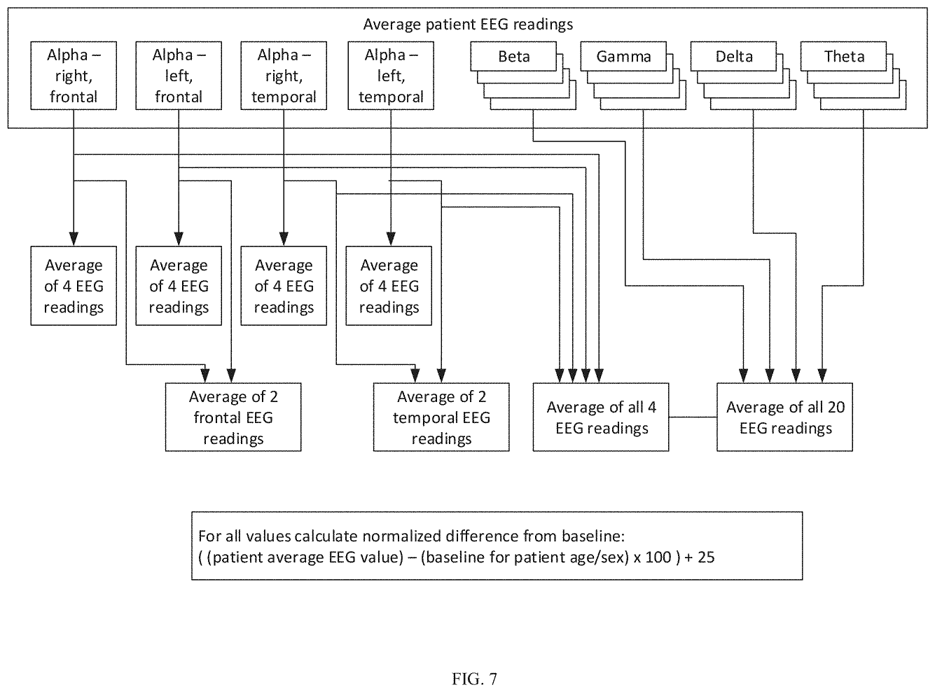

In step 508, a number of further average values are derived and calculated from the average values of step. These derived average values are illustrated in FIG. 7 and include:
1. The average of the average EEG reading for each EEG wave type from the four electrodes. For example, the average of the delta wave type EEG readings from each of the right front, left front, right temporal, and left temporal electrodes.
2. The difference between the average EEG readings for the front electrodes for each EEG wave type. For example, the difference between the average readings from the right frontal electrode and average readings from left frontal electrode.
3. The difference between the average EEG readings for the temporal electrodes for each EEG wave type. For example, the difference between the average readings from the right temporal electrode and average readings from left temporal electrode.
4. The average of the differences between the right and left front electrode EEG average readings for all wave types.
5. The average of the differences between the right and left temporal electrode EEG average readings for all wave types.
6. The average of all average EEG readings from all electrodes and for all wave types.

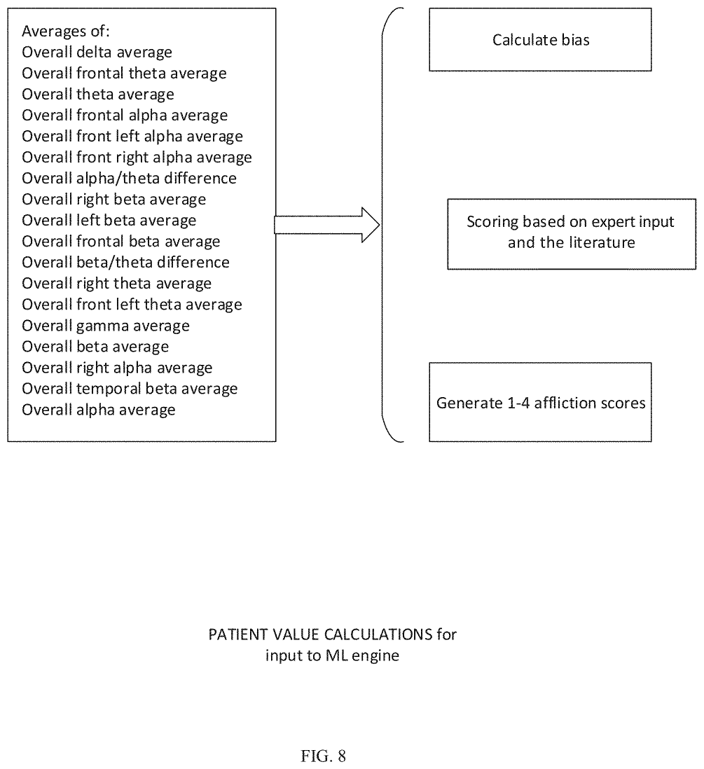

In step 510, the valued calculated in steps 506 and 508 are used to derive 18 pre-final score specific averages of the EEG values that may be correlated with the medical conditions of interest as shown in FIG. 8. Scoring may be based on the literature combined with expert opinions. Each medical condition has its own algorithm that utilizes a subset that is a weighted combination of the 18 pre-final scores. For example, of the 18 pre-final scores, according to the literature only four variables are primarily involved in an OCD diagnosis. For depression there can be up to eight variables involved and they may include some of the same variables as OCD. Each algorithm includes calculating a weighted average of the relevant pre-final score percentages. The weighting for each pre-final score may be based on the importance of each pre-final score to predicting the risk of developing the medical condition. For each condition of interest, the pre-final scores to be combined and the weights may be predefined based on knowledge of how each variable predicts the condition as known in the art. These pre-final scores include:
7. The average value of all average delta wave type readings to indicate elevated delta wave activity.
8. The average value of all average theta wave type readings measured by the right and left frontal EEG electrodes to indicate higher frontal theta wave activity.
9. The average value of all average theta wave type readings to indicate theta wave activity.
10. The average value of all average alpha wave type readings measured by the right and left frontal EEG electrodes to indicate frontal alpha wave activity.
11. The average value of all average alpha wave type readings measured by the right frontal EEG electrodes.
12. The average value of all average alpha wave type readings measured by the left frontal EEG electrodes.
13. The difference between the average of the alpha wave type average measurements and the average of the theta wave type right average measurements and front left average measurements. This provides an indication of the relationship between alpha and theta wave types in the patient.
14. The average value of all average beta wave type readings measured by the right EEG electrodes.
15. The average value of all average beta wave type readings measured by the left EEG electrodes.
16. The average value of all average alpha wave type readings measured by the left frontal and right frontal EEG electrodes.
17. The difference between the average of the beta wave type average measurements and the average of the theta wave type measurements. This provides an indication of the relationship between beta and theta wave types in the patient.
18. The average value of all average theta wave type readings measured by the right EEG electrodes.
19. The average value of all average theta wave type readings measured by the left EEG electrodes.
20. The average value of all average gamma wave type readings to indicate elevated gamma wave activity.
21. The average value of all average beta wave type readings to indicate elevated beta wave activity.
22. The average value of all average alpha wave type readings measured by the right frontal and left frontal EEG electrodes to indicate higher frontal alpha wave activity.
23. The average value of all average beta wave type readings measured by the right temporal and left temporal EEG electrodes to indicate higher frontal beta wave activity.
24. The average value of all average alpha wave type readings to indicate elevated alpha wave activity.

All of these values are computed for the patient EEG data as well as for the baseline values for each of the age/sex groups. In step 512, the patient EEG data is compared to the baseline values for the patient's age/sex group. For each of the calculated values of step 510, a normalized difference is calculated by subtracting the baseline value from the patient's EEG value and normalizing to be a positive real number between 0 and 100. In embodiments, the difference may be multiplied by 100 then has 25 added to the resulting value. The value is then further divided by 25 and expressed as a percentage.

In step 514 a final EEG score is calculated for each medical condition of interest based on the valued of step 512. It is known in the medical literature that there is a correlation between some medical conditions and EEG readings. For example, the risk of developing OCD is correlated with elevated delta waves, high frontal alpha waves, high right beta waves, and elevated right theta waves. Some learning and memory problems are correlated with elevated delta waves, elevated front left theta waves, below average gamma waves, and high beta waves.

In step 516, for each medical condition a BIAS condition is evaluated to determine if there is a statistically significant probability a patient developing that medical condition. If the BIAS condition is met, a probability value is evaluated. Separate BIAS conditions are calculated for each medical condition of interest. Algorithms to calculate BIAS conditions are formulated based on human review of each medical condition's literature to use pre-final score specific averages of the EEG values to predict medical conditions. These algorithms may be adjusted as needed based on trial and error testing. The pre-final scores are reflected in the reference baseline values for each age and sex combination. Each reference baseline corresponds to pre-final scores that generate no increase in the probability of a patient developing the medical condition in question.

The BIAS conditions algorithms may be tested using a method with a training set that includes pre-final scores representing all age/sex categories, with baseline pre-final scores removed. The baseline pre-final scores are stripped of their EEG data and a modified interpreted algorithm is used to predict the pre-final scores. If the algorithm predicts values for the blank pre-score fields, the associated pre-final scores are analyzed to determine the cause and adjust average value calculation(s) so that the interpreted algorithm does not produce any non-zero pre-score fields. Once an interpreted algorithm is found that does not fill in any blank pre-final score fields, a new training set may be generated by applying the algorithm to all pre-final scores in the reference database until the new training set is completed. As new pre-final scores are added over time, the method may be repeated to update the training set.

In embodiments, a test batch of records can be used to test multiple machine algorithms to determine which algorithm generate the smallest differentials between pre-machine values and machine generated values. A test batch of records is created by selecting a number of non-baseline pre-final scores for a single age/sex group and remove all pre-scoring values from the RRs. Each machine algorithm is then tested to determine which one(s) generate the smallest differentials between the pre-machine values and machine generated values. The filled-in values represent probable pre-score quantification that can be charted or otherwise presented.

In step 518, the calculated BIAS condition for each medical condition is converted into a final score value between 0 and 4 with 0 meaning that it is “unlikely” and 4 meaning that it is “highly probable” that the patient will experience the particular condition. Final scores may be used to show how the degree that a patient's results may deviate from the baseline readings for their age/sex group for a particular medical condition.

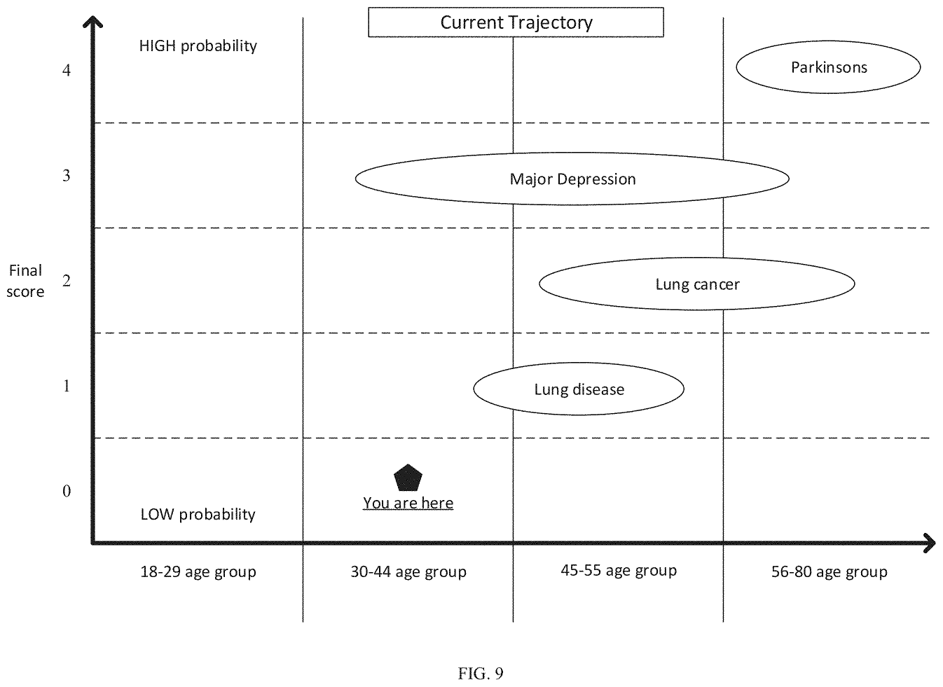

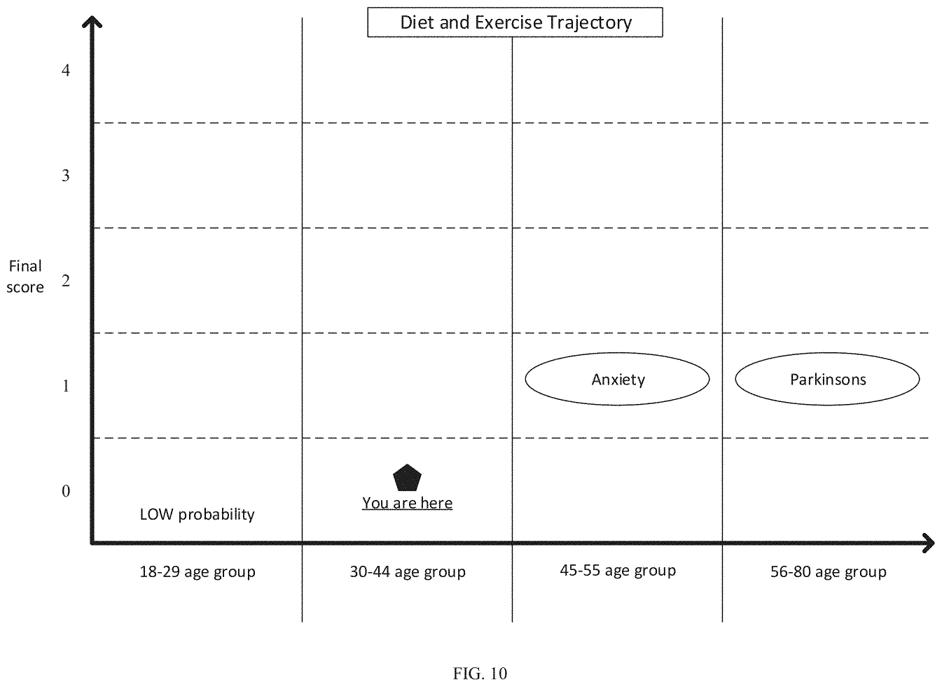

The steps of 512, 516, and 518 may be repeated for multiple baseline values to provide a timeline of future risk as a patient ages. For example, if a patient is 35 years old, they may be evaluated against baseline value for the 30-44 age group in step 512 to obtain risk probabilities for the 30-44 age group. The same patient may then also be evaluated against the baseline values of the 45-55 age group to obtain risk probabilities for when the patient reaches this age. Similarly, the same patient may then also be evaluated against the baseline values of the 56-80 age group to obtain risk probabilities for when the patient reaches this age. The risk probabilities for the multiple age groups may be combined to provide a timeline as part of the patient report of step 520 and as illustrated in FIG. 9 and FIG. 10.

In optional step 520, reports may be generated for the patient, their caregiver, medical professional, or other authorized person. In embodiments, a wide variety of reporting methods and data export methods may be used.

FIG. 9 illustrates an example of a patient report as generated by embodiments. Based on a patient evaluation using method 200 it is found that the patient, who is in the 30-44 age has elevated risk of developing some medical conditions in the future based on their current trajectory as indicated by their biometric questionnaire responses. In this illustration, the patient has elevated risks of developing major depression before the age of 55, a high probability of developing Parkinson's disease between the ages of 56 and 80, a medium risk of developing lung cancer between the ages of 45 and 80, and a lower probability of developing lung disease before the age of 55. The patient report of FIG. 9 may be accompanied by suggestions on how the patient may adjust their lifestyle in order to reduce the risk of developing the indicated medical conditions. The age categories correspond to the age groupings of the baseline calculations illustrated in FIG. 3 and different embodiments may use more or less grouping and use different ages.

FIG. 10 illustrates an example of a patient report as generated by embodiments for the patient of FIG. 9 assuming they implement some or all of the suggestions that accompanied the patient report of FIG. 9. In this case, it indicates that the patient still has a low probability of developing an anxiety conditions between the ages of 45-55 and of Parkinson's disease between the ages of 56-80. The age categories correspond to the age groupings of the baseline calculations illustrated in FIG. 3 and different embodiments may use more or less grouping and use different ages.

Returning to FIG. 4, in step 410, correlations are established between the biometric data (from the questionnaire) and the FES numbers (from the EEG), and the correlations are ordered by degree of overlap. New questionnaire answers are correlated with the training set and in step 410, a predicted probability of developing a medical condition (i.e., a mental affliction) is generated. In step 412, the predictions are added to the training database. Given 4 age groups and two sexes, results for a number of people of the same sex with similar biometrics but different age groups may be appended to produce FES prediction results for a longer timeline, such as a 60 year timeline from 20 to 80 years of age. In embodiments, the beginning of the timeline is determined by the patient's age group while the ending of the timeline is determined by the oldest age group for which baseline data is available.

Once sufficient data is obtained for each age and sex group, a final trained system is obtained and may be used by a patient to predict their chance of developing a particular medical condition at various stages of their life. The patient may then choose to alter their lifestyle, thereby affecting their responses to the biometric questionnaire, and reduce their chances of developing any or all of the predicted medical conditions.

In use, the person inputs data to the questionnaire 102, the AI/ML model 104 correlates the questionnaire responses with the correct training set for the patient's age and sex, and a report may be generated for the patient. The report can show a timeline of predicted illness or condition onsets and may suggest various lifestyle changes to move the illness or condition probability toward baseline “normal”.

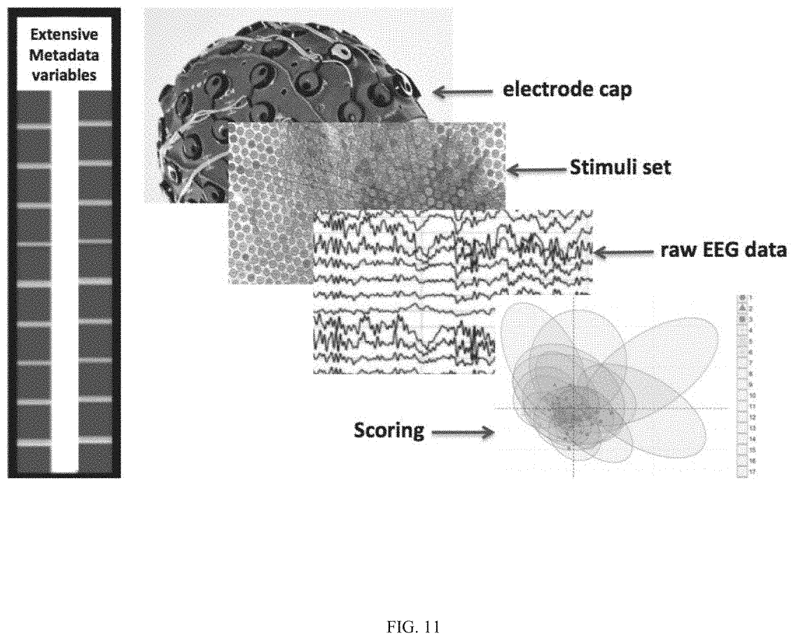

FIG. 11 depicts the components of the validated information collection according to an embodiment. Metadata consists of results of a biometric survey related to a patient's situation. Electrode cap is used to gather raw EEG data when the patient is subjected to a specified set of Stimuli. Raw EEG data is digitized as x-y coordinates and is correlated with the biometric results by an ML engine to produce a probability of developing a medical condition.

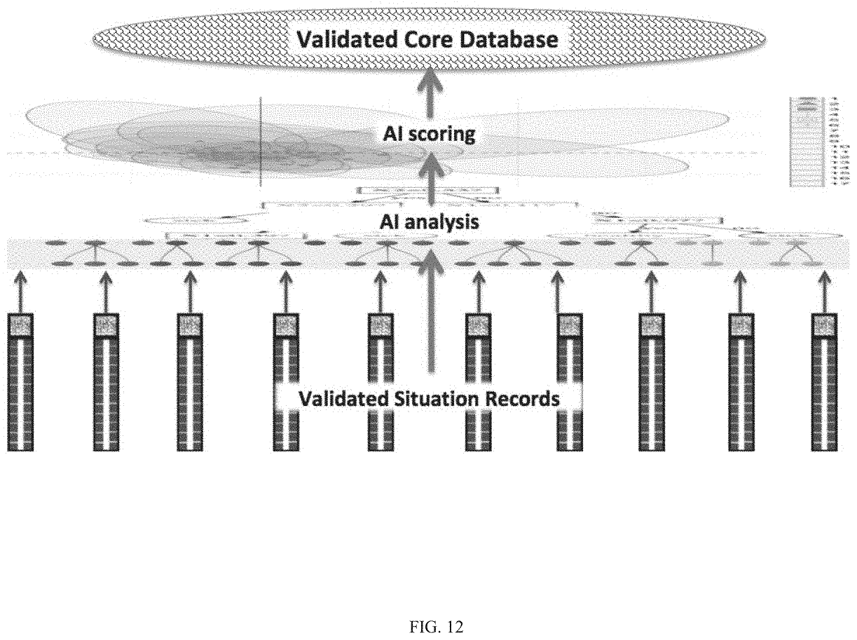

FIG. 12 depicts and embodiment illustrating how scored records including both EEG, predicted or real, and biometric data may be subjected to an artificial intelligence-based ML engine that generates results that are re-processed by artificial intelligence-based scoring algorithm for each medical condition. The re-processed data may then be then added to the validated core database.

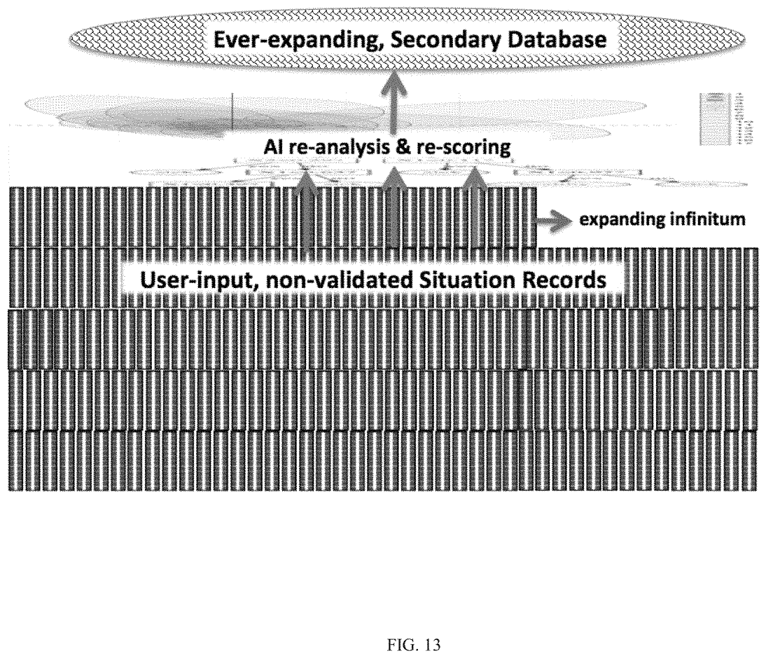

FIG. 13 depicts an embodiment where a number of incoming records are input to an artificial intelligence/machine engine for re-analysis and re-scoring to be added to a secondary database.

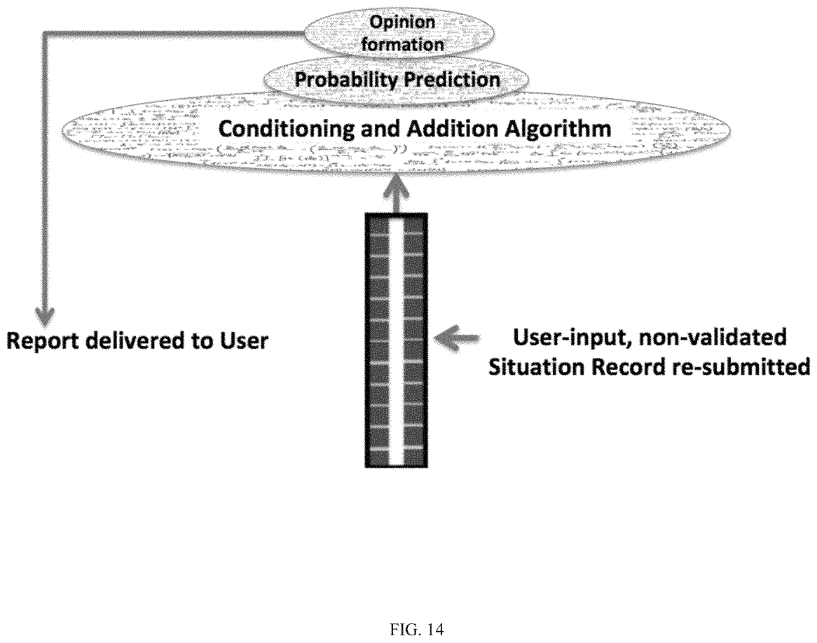

FIG. 14 depicts an embodiment where an individual record being resubmitted to a conditioning and addition algorithm, which may be artificial intelligence-based, and the output being processed by probability prediction algorithm, which may be artificial intelligence-based, and whose output is subjected to an opinion formation algorithm 16, that also may be artificial intelligence-based.

It will be appreciated that, although specific embodiments of the technology have been described herein for purposes of illustration, various modifications may be made without departing from the scope of the technology. The specification and drawings are, accordingly, to be regarded simply as an illustration of the invention as defined by the appended claims, and are contemplated to cover any and all modifications, variations, combinations or equivalents that fall within the scope of the present invention. In particular, it is within the scope of the technology to include a computer program product or program element, or a program storage or memory device such as a magnetic or optical wire, tape or disc, or the like, for storing signals readable by a machine, for controlling the operation of a computer according to the method of the technology and/or to structure some or all of its components in accordance with the system of the technology.

Although the present invention has been described with reference to specific features and embodiments thereof, it is evident that various modifications and combinations can be made thereto without departing from the invention. The specification and drawings are, accordingly, to be regarded simply as an illustration of the invention as defined by the appended claims, and are contemplated to cover any and all modifications, variations, combinations, or equivalents that fall within the scope of the present invention.

What is claimed is:

1. A method for calculating a probability of developing a medical condition, the method comprising:
receiving results of a biometric questionnaire of a patient;
providing the results to a machine learning (ML) engine, the ML engine correlating the results with a training set to produce correlated results;
receiving the correlated results from the ML engine, the correlated results predicting a probability of the patient developing the medical condition.

2. The method of claim 1 wherein the probability is calculated by comparing EEG estimates to baseline values.

3. The method of claim 2 wherein the baseline values are selected based on the patient's age, sex or other biometric variables.

4. The method of claim 3 further comprising calculating a second probability of the patient developing the medical condition, the second probability based on comparing the EEG estimates to baseline values corresponding to a future age of the patient, the second probability predicting a risk of the patient developing the medical condition at a future time.

5. The method of claim 4 further comprising using the probability and the second probability to produce a timeline of a risk of the patient developing the medical condition over a time span.

6. The method of claim 1 further comprising generating a report indicating the probability of the patient developing the medical condition at a future date.

7. The method of claim 6 wherein the report provides information to allow the patient to make biometric changes to reduce the probability of the patient developing the medical condition at a future date.

8. A method of training a machine learning (ML) engine, the method comprising:
receiving biometric results of a biometric questionnaire of a patient;
receiving electroencephalogram (EEG) results of the patient;
trimming the EEG results to obtain stable EEG results;
converting the EEG results into a plurality of summary values;
scoring the plurality of summary values to produce a record;
providing the record to a machine learning (ML) engine;
receiving predictions of a medical condition from the ML engine;
comparing the predictions of a medical condition to scoring predictions to generate an error; and
utilizing the error to adjust coefficients of the ML engine.

9. The method of claim 8 further comprising repeating the method until the error is below a threshold.

10. The method of claim 8 wherein the plurality of summary values are used to produce baseline values when the ML engine is used in a prediction mode.

11. The method of claim 10 wherein the baseline values are associated with an age, a sex, or another biometric variable of the patient.

12. The method of claim 8 wherein the trimming of the EEG results comprises discarding a last portion of the EEG results and discarding a first portion of the remaining EEG results.
